(19) United States
(12) Patent Application Publication
(10) Pub. No.: US 2005/0163734 A1
(43) Pub. Date: Jul. 28, 2005

(54) Cosmetic composition having whitening effect comprising extract of pulsatilla radix as main ingredient

(76) Inventors: Jong Seok Kim, Gongju-Si (KR); Hye Young Kim, Gongju-Si (KR); Jong Uk Kim, Gongju-Si (KR)

Correspondence Address: BIRCH STEWART KOLASCH & BIRCH, PO BOX 747, FALLS CHURCH, VA, 22040-0747, US

(21) Appl. No.: 11/086,919

(22) Filed: Mar. 23, 2005

Related U.S. Application Data
(63) Continuation of application No. PCT/KR03/01905, filed on Sep. 18, 2003.

(30) Foreign Application Priority Data
Sep. 23, 2002 (KR) 10-2002-0057653

(51) Int. Cl. A61K 35/78; A61K 7/135

(52) U.S. Cl. 424/62; 424/773; 424/728; 424/757; 424/769; 514/61; 514/453

(57) ABSTRACT

Disclosed is whitening cosmetic composition comprising extract of Pulsatillae Radix as main ingredient and if necessary, additionally comprising ingredient(s) selected from the group consisting ranunculin, deoxypodophyllotoxin and 3-O-α-L-ramnopyranosyl(1→2)-[β-D-glucopyranosyl(1-4)]-α-L-arabinopyranoside(SB365) obtained from the extract of Pulsatillae Radix and auxiliary ingredient(s) selected from the group consisting extract of Ulmaceae Cortex and extract of Glycyrrhizae Radix. The present composition has an excellent whitening effect.

### TECHNICAL FIELD

The present invention relates to a cosmetic composition comprising extract of Pulsatillae Radix as main ingredient.

The skin of human being consisted of epidermis and dermis. Nails and hairs are formed in the epidermis and sweat glands exist there too. In dermis there exist nerve nets, blood vessels and sweat glands. Melanin-producing—melanocytes exist in epidermis. The starting material for producing melanin is tyrosine, an essential amino acid and the tyrosine is oxidized by tyrosine hydroxylase(TH) into 3,4-dihydroxyphenylalanine(DOPA) and the DOPA is experienced through several reaction steps to be changed into melanin, a black polymer. If the TH-function is congenitally deficient, albinism is appeared.

Accordingly, The most important target of development of whitening material in biosynthetic mechanism of melanin is to find any material which prevents the activity of tyrosine hydroxylase.

Intensive studies have been being focused to find tyrosine hydroxylase inhibitor from natural products. Korean Patent Laid-open Publication No. 2002-0023168 discloses that the extract of Gardeniae fructus has inhibitory effect against tyrosine hydroxylase by 3 times than arbutin in comparative test. However, as the said patent specification did not disclose which material in the extract of Gardeniae fructus has such inhibitory action against tyrosine hydroxylase, it could not evaluate whether the extract of Gardeniae fructus has toxic effect on the skin of human being or not. Until now, materials such as dihydroxybenzene derivatives, retinoid series and steroid hormones had been being used as tyrosine hydroxylase inhibitor. However, the said materials showed to have side effects on the skin and therefore the use of the said materials is restricted.

### BACKGROUND ART

The present invention relates to a whitening cosmetic composition comprising extract of Pulsatillae Radix as main ingredient.

The present invention relates to a whitening cosmetic composition comprising extract of Pulsatillae Radix and one or more ingredient(s) selected from the group consisting ranunculin, deoxypodophyllotoxin and 3-O-α-L-ramnopyranosyl(1→2)-[β-D-glucopyranosyl(1→4)]-α-L-arabinopyranoside(SB365) extracted and isolated from the extract of Pulsatillae Radix as main ingredients.

The present invention relates to a whitening cosmetic composition comprising extract of Pulsatillae Radix and extract of bark of Ulmus macrocarpa as main ingredients.

### DISCLOSURE OF INVENTION

One object of the present invention is to provide a whitening cosmetic composition comprising the extract of Pulsatillae Radix as main ingredient.

The other object of the present invention is to provide a whitening cosmetic composition comprising the extract of Pulsatillae Radix and the extract of the bark of the Ulmus macrocarpa as main ingredients.

Another object of the present invention is to provide a whitening cosmetic composition comprising the extract of Pulsatillae Radix and one or more ingredient(s) selected from the group consisting ranunculin, deoxypodophyllotoxin and 3-O-α-L-ramnopyranosyl(1→2)-[β-D-glucopyranosyl(1→4)]-α-L-arabinopyranoside(SB365) extracted and isolated from the extract of Pulsatillae Radix as main ingredients.

Still another object of the present invention is to provide a whitening cosmetic composition comprising the extract of Pulsatillae Radix as main ingredient and more comprising one or more extracts selected from the group consisting the extract of the bark of the Ulmus macrocarpa, extract of Ginseng Radix and extract of Glycyrrhizae Radix as auxiliary ingredients.

Still another object of the present invention is to provide a whitening cosmetic composition comprising the extract of Pulsatillae Radix and one or more ingredient(s) selected from the group consisting ranunculin, deoxypodophyllotoxin and 3-O-α-L-ramnopyranosyl(1→2)-[β-D-glucopyranosyl(1→4)]-α-L-arabinopyranoside(SB365) extracted and isolated from the extract of Pulsatillae Radix as main ingredients and more comprising one or more extracts selected from the group consisting the extract of the bark of the Ulmus macrocarpa, the extract of Ginseng Radix and the extract of Glycyrrhizae Radix as auxiliary ingredients.

Pulsatillae Radix is the root of Pulsatilla koreana, P. cerna and P. chinensis, etc. The Pulsatillae Radix has been being used as oriental medicine for treating child bed fever, detoxication, antidiarheal, bactericide, amebicide, fungicide, etc. Recently, the Pulsatillae Radix is reported to have antitumor activity and is now under clinical study.

The bark of Ulmus marocarpa has being been used as oriental medicine for treating various parasites, various kinds of epidermophytid(Oriental Materia Medica, H-Y Hsu et. al., Oriental Healing Arts Institute, pp 749). Therefore, the bark of Ulmus marocarpa is regarded to have protecting effect for the skin.

Deoxypodophyllotoxin isolated from the extract of Pulsatillae Radix is known compound and has the following structural formula.

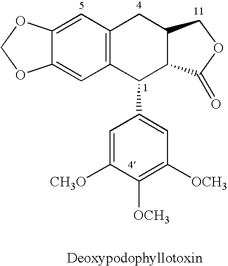

Ranunculin isolated from the extract of Pulsatillae Radix is known compound and has the following structural formula.

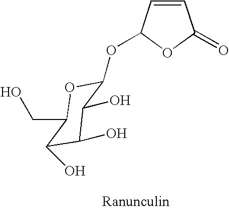

3-O-α-L-ramnopyranosyl(1→2)-[β-D-glucopyranosyl(1→4)]-α-L-arabinopyranoside(SB365) of which generic name is hederagenin, which isolated from the extract of Pulsatillae Radix is known compound and has the following structural formula.

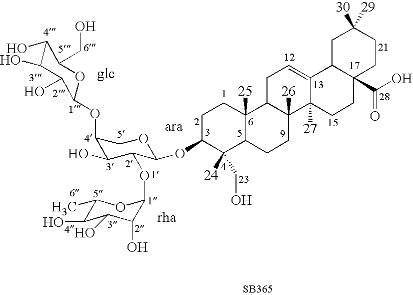

The said compounds are confirmed to have excellent whitening effects by the present invention.

The present whitening composition can be formulated by adding conventional vehicles to cream, injection, capsule, tablet, etc., by conventional preparation methods.

Extracting solvent such as water; lower alkanol e.g. methanol, ethanol, propanol or butanol; methylenechloride; acetone; or mixture thereof can be used in the present invention.

### BRIEF DESCRIPTION OF DRAWINGS

FIG. 1 indicates the results of chromatogram of PT fraction through Sepadex LH20 column chromatography.

### BEST MODE FOR CARRYING OUT THE INVENTION

The present invention is explained in more detail by the following examples and experimental examples.

### EXAMPLE 1

General Preparation of the Extract of Pulsatilla Radix

The Pulsatillae Radix were powdered to obtain powder of 40-100 mesh. A certain volume of the powder was put in an extractor and extraction was carried out with lower alcohol-water mixed solvent. Lower alcohol is selected from the group consisting methanol, ethanol, propanol and butanol. Etanol-water mixed solvent among them is the best selectivity in extraction. In the case of the use of ethanol-water mixed solvent of 50% (v/v), materials having very big polarity and materials of large molecular weight and polymers, etc. were not extracted. Extraction were carried out under the temperature of 15° C.˜35° C., most preferably 20° C.˜25C. Extraction time carried out from 1 hour unit and gradually increased. Extraction time of 3hours is preferable. The extract which Pulsatillae Radix was extracted by using 50% aqueous ethanol solvent under the temperature of 25° C. for 3 hours is expressed as ET fraction and the ET fraction was used for measuring the whitening effect.

### EXAMPLE 2

1. Isolation of Materials Having Whitening Effect.

The whitening effect was confirmed by clinical trial with a group of human beings of the following experimental examples. The isolation of materials having whitening effect was carried out by the method of measurement for tyrosinase-inhibition effect which is generally used.

Low molecular, polar materials were extracted from acetone and the acetone-extract was expressed as PA fraction and the remaining part was expressed as PT fraction.

PT and PA fractions showed respectably 10% and 73% inhibition effects against tyrosinase.

2. Active Material 1 of the PT Fraction

The fraction 3 which was obtained by re-fractionation of the PT fraction with Sephadex was refined to obtain an active ingredient which was ascertained as 3-O-α-L-ramnopyranosyl(1→2)-[μ-D-glucopyranosyl(1→4)]-α-L-arabinopyranoside(SB365) of which generic name is hederagenin. This compound is ascertained to have antitumor activity(Now patent pending). This material showed no effect against tyrosinase but showed an excellent whitening effect in clinical trials. So, this material is sure to have whitening effect through another mechanism. It is to be assumed that when relation between saponin derivative and the surface of skin cell is to be thought, the compound hinders the migration of melanosome and prevents melanin to be accumulated on keratinocyte.

3. Whitening Material 2 of the PT Fraction

The SPX2 fraction which was obtained by fractionation of the PT fraction by Sephadex showed inhibition of 48% against tyrosinase. A material which was obtained by passing the SPX2 fraction through silicagel column (Eluting solvent:methylenechloride/methanol=4:1) and showed Rf=0.3 was confirmed. The Rf value was identified as those of ranunculin. The NMR data of the material which was isolated by a known method was the same with those of the ranunculin. Ranunculin is broadly spread in plants which belonged to Ranunculaceae and is known to have mitotoxicity (Vonderbank, Pharmazie 5, 21(1950)). Pure ranunculin showed inhibition of 51 % against tyrosinase.

4. Active Material of the PA Fraction

Deoxypodophyllotoxin is spread in various plants including Anthriscus sylvestris Hoffm and shows to have mitotoxicity (Byung-Zun Ahn, Song-Bae Kim, Yong Kim, et al. Use of Deoxypodophyllotoxin as antitumor agent, Korean Patent No. 315,200). It is reported that this material inhibits the formation of blood vessel of endotheliocyte of umbilicus in human being(Yong Kim, Song-Bae Kim, Byung-Zun Ahn, et al., Deoxypodophyllotoxin; the cytotoxic and antiangiogenic component from Pulsatilla koreana Nakai, Planta Medica, 68, 271-274(2002)). This material exhibits inhibition of 38% at 0.03 μg/ml against tyrosinase. It was difficult to experiment at higher concentration because this material has low water solubility.

### EXAMPLE 3

50 g of dried finely powdered Pulsatilla Radix and 500 ml of 50% ethanol were added to an extractor. The mixture was stirred at room temperature for 3 hours and filtered. The filtrate was stored and the procedure was carried out with the remaining residue for 2 times. The combined filtrate was concentrated under reduced pressure and dried to obtain 23 g of extract.

### EXAMPLE 4

PT Fraction Having Inhibition Against Tyrosinase and Preparation of the PA Fraction

20 g of the extract obtained in Example 3 was added to 200 ml of acetone. The mixture was stirred for 10 minutes to obtain a suspension. The suspension was filtered to obtain solution and residue. The residue was suspended in 200 ml of acetone and stirred for 10 minutes and filtered. The residue was dried to obtain PT fraction (17.4 g). The combined solution was concentrated and dried to obtain PA fraction (3.5 g).

### EXAMPLE 5

Isolation of PTpur, an Inhibitor Material Against Tyrosinase from PT Fraction

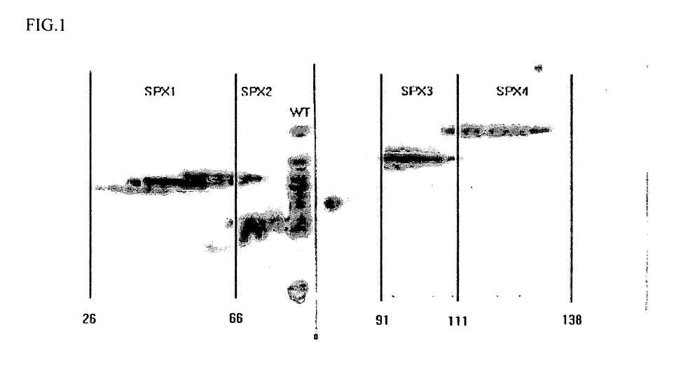

560 mg of the PT fraction was eluted and fractionated through Sephadex LH20 column (200 g, 60×4 cm) (velocity of elution: 1 ml/1 min.). Fractionation was carried out and filled 0.5 ml of volume of the fractionation per 1 test tube by use of test tubes. These fractions were in turn spotted on thin layers of silica gel and developed for fractions to be divided(Developing solvent:butanol:acetic acid:water=4:1:1, coloration:spraying of sulfuric acid and heated). The results were showed in the FIG. 1.

In the FIG. 1, the PT1 fraction (139 mg, 24.8%) was collected from the test tube Nos. 26˜66 and main spots are 4. The lower parts were reacted with sulfuric acid to appear yellow color.

The PT2 fraction (344 mg, 61.4%) was collected from the test tube Nos. 66˜91 and main sports are 2.

The PT3 fraction (61 mg, 10.9%) was collected from the test tube Nos. 91˜111. When spraying of sulfuric acid and heating, firstly red color was appeared and the time being, blue color appeared. The fraction is the main material of which spots existed between Rf value is in mean 0.48˜0.50.

The PT4 fraction (15.7 mg, 2.8%) was collected from the test tube Nos. 111˜138.

The SPX3 fraction and the SPX4 fraction were appeared each one spot on the thin layers and were fractions which were relatively pure. The fractions were shown on the FIG. 1.

The SPX1, SPX2, SPX3 and SPX4 fractions were obtained through Sephadex LH20 column chromatography.

The SPX3 fraction was relatively pure fraction and was obtained from white precipitation which the fraction was dissolved in 0.5 ml of water and stored to precipitate (PTpur).

### EXAMPLE 6

Identification of the PTpur Structure

As the above isolated material PTpur has the identifying data, that is, m.p. 239˜241° C., [α]_{D}=+23.6° (c, 0.2, MeOH), white amorphous, and positive in Riberman-Bucart Reaction is identified as a glycoside. In addition, as the material has IR (cm⁻¹) data, 3400(br, —OH), 2940(br, C—H), 1695(C═O), 1455, 1040(C—O) and adsorption band of 1000-1100, 3000-3400 or more, the material was largely regarded as a possibility of glycoside.

¹H-NMR of the material follows the typical pattern of those of saponin and 6-CH₃ groups were observed at 0.91, 0.92, 0.98, 1.00, 1.07 and 1.21 ppm and another —CH₃ group was observed at 1.64 ppm as doublet. From these, one saccharide among consisting saccharides is assumed as rhamnose. Anomeric proton was observed at 6.25(br.), 5.11(1H, J=7.80 Hz) and 4.97 ppm (1H, J=6.66 Hz). Therefore, PTpur was identified as a glycoside bonded 3 saccharides.

From ¹³C-NMR, hydroxymethyl group was observed at 65.4 ppm(C-23); 3 anomeric carbon signals were observed respectively at 140.2(C-1′), 106.7(C-1′″) and 101.7 ppm(C-13); 2 olefin carbons were respectively at 122.5 ppm(C-12) and 144.8 ppm(C-13); a carboxyl carbon was observed at 180.2 ppm(C-28). Conventionally, when a saccharide is bonded at C-28, glycosylation upfield shift of about 4 Hz is observed. But, such shift was not observed on this compound. Therefore, It could be ascertained that this compound is not glycoside which saccharide was not bonded at C-28.

And then, for identifying the structure of aglycone, this compound was hydrolyzed by ethanol/sulfuric acid. When physical and chemical data, ¹³C-NMR and ¹H-NMR data of the hydrolyzed product were compared, it was ascertained that PTpur was hederagenin. The hydrolyzed saccharides were ascertained as rhamnose, arabinose and glucose by comparative TLC.

When generally analyzing the above results and data published in arts, PTpur is ascertained to be 3-O-α-L-ramnopyranosyl(1→2)-[β-D-glucopyranosyl(1→4)]-α-L-arabinopyranoside(SB365) of which generic name is hederagenin, a saponin already isolated from the Pulsatilla Radix.

¹H-NMR and ¹³H-NMR data of the PTpur showed in the following table.

TABLE 1

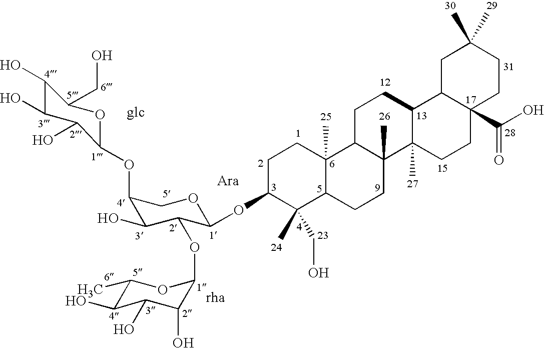

| position | ¹H (ppm) | J (Hz) | ′″C (ppm) | position | ¹H (ppm) | J (Hz) | ′″C (ppm) |
|---|---|---|---|---|---|---|---|
| C-1 | | | 36.9 | Arabinose | | | |
| C-2 | | | 25.1 | C-1′ | 4.97 d | 6.66 | 140.2 |
| C-3 | 3.28 d | 10.9 | 81.0 | C-2′ | | | 80.4 |
| C-4 | | | 43.5 | C-3′ | | | 75.4 |
| C-5 | | | 48.1 | C-4′ | | | 76.2 |
| C-6 | | | 18.1 | C-5′ | | | 63.9 |
| C-7 | | | 32.8 | Rhamnose | | | |
| C-8 | | | 39.7 | C-1″ | 6.25 br | | 101.7 |
| C-9 | | | 47.8 | C-2″ | | | 72.3 |
| C-10 | | | 36.9 | C-3″ | | | 72.4 |
| C-11 | | | 22.9 | C-4″ | | | 74.1 |
| C-12 | 6.46 s | | 123.5 | C-5″ | | | 69.6 |
| C-13 | | | 144.8 | C-6″ | 1.64 | 6.94 | 18.6 |
| C-14 | | | 42.1 | Glucose | | | |
| C-15 | | | 28.3 | C-1′″ | 5.11 d | 7.80 | 106.7 |
| C-16 | | | 23.8 | C-2′″ | | | 75.0 |
| C-17 | | | 46.2 | C-3′″ | | | 78.5 |
| C-18 | | | 41.9 | C-4′″ | | | 71.2 |
| C-19 | | | 46.4 | C-5′″ | | | 78.8 |
| C-20 | | | 30.9 | C-6′″ | | | 62.5 |
| C-21 | | | 34.2 | | | | |
| C-22 | | | 33.2 | | | | |
| C-23 | 4.36, 3.67 | overlap | 65.4 | | | | |
| C-24 | 1.07 s | | 14.0 | | | | |
| C-25 | 0.91 s | | 16.0 | | | | |
| C-26 | 0.98 s | | 17.4 | | | | |
| C-27 | 1.21 s | | 26.3 | | | | |
| C-28 | — | 180.2 | | | | | |
| C-29 | 0.92 s | | 32.8 | | | | |
| C-30 | 1.00 s | | 23.7 | | | | |

### EXAMPLE 7

Isolation of PTculin, an Inhibitor Material Against Tyrosinase from the PT Fraction

When the PT fraction and pure ranunculin were developed on thin layer of silica gel, the same spots were observed at Rf=0.3.
(Solvent: methylenechloride/methanol=4:1)

### EXAMPLE 8

Isolation of PApur, an Inhibitor Material Against Tyrosinase from the PA Fraction

30 mg of PA fraction was dissolved in 2 ml of methanol and 3 ml of hexane was added thereto and the mixture was stirred. After standing a while, hexane layer was decanted. Such extraction was carried out 2 times. The combined hexane solution was dried and carried out a comparative chromatography with deoxypodophyllotoxin on thin layer of silica gel. It was ascertained that the same spot with the deoxypodophyllotoxin was observed from the hexane solution. The spot was isolated from fractionating silica gel thin layer to obtain 3 mg of deoxypodophyllotoxin which was the same with the standard of the deoxypodophyllotoxin when NMR was measured.

The present invention is described in more detail with the following preparative examples.

Preparative Example 1
Emulsion

| Composition of emulsion base | (g) |
|---|---|
| Cetyl octanoate | 4.0 |
| Cyclomethicone | 3.0 |
| Fluid paraffin | 4.0 |
| Polysorbate 60 | 5.0 |
| Sorbitan stearate | 3.0 |

To the each bases(each 19 g), each 100 mg, 400 mg, 600 mg, 800 mg and 1000 mg of the ET fraction were added and mixed uniformly to obtain each preparation P100, P400, P600, P800, P1000 mg.

Preparative Example 2
Injection

Pulsatilla Radix as main ingredient, bark of Ulmus macrocarpa, Ginseng Radix, Glycyrrhizae Radix as auxiliary ingredients were extracted with 50% ethanol. The extract was prepared, filled in vials and lyophilized to obtain injections by conventional preparation method of injections.

Preparative Example 3

| | |
|---|---|
| Composition of Example 3 | 100 mg |
| Starch | 100 mg |
| Lactose | 50 mg |
| Magnesium stearate | q.s. |

The above ingredients were prepared to obtain a capsule by a conventional preparation method of capsules.

Preparative Example 4

| | |
|---|---|
| Composition of Example 3 | 100 mg |
| Lactose | 50 mg |
| Magnesium stearate | q.s. |
| Talc | q.s. |

The above ingredients were prepared to obtain a tablet by a conventional preparation method of tablets.

Preparative Example 5

| Composition of Example 3 | 3.0 | g |
|---|---|---|
| Isomerized saccharide | 50.0 | g |
| Sodium alginate | 50.0 | mg |
| Sodium benzoate | q.s. | |
| Purified water | q.s. to 100 | ml |

The above ingredients were prepared and filled in brown bottle of 100 ml to obtain solution by a conventional preparation method of solution.

Preparative Example 6

| Injection | | |
|---|---|---|
| Composition of Example 3 | 100 | mg |
| Ranunculin | 10 | mg |
| Water for injection | q.s. to 1 | ml |

The above ingredients were prepared and filled in ampoule of 1 ml to obtain injection by a conventional preparation method of injection.

Preparative Example 7

| | |
|---|---|
| Composition of Example 3 | 100 mg |
| Deoxypodophyllotoxin | 10 mg |
| SB 365 | 10 mg |
| Starch | 100 mg |
| Lactose | 50 mg |
| Magnesium stearate | q.s. |

The above ingredients were prepared to obtain a capsule by a conventional preparation method of capsules.

Preparative Example 8

| | |
|---|---|
| Composition of Example 3 | 100 mg |
| Deoxypodophyllotoxin | 10 mg |
| Lactose | 50 mg |
| Magnesium stearate | q.s. |
| Talc | q.s. |

The above ingredients were prepared to obtain tablet by a conventional preparation method of tablets.

Preparative Example 9

| Composition of Example 3 | 3.0 | g |
|---|---|---|
| SB 365 | 100 | mg |
| Isomerized saccharide | 50.0 | g |
| Sodium alginate | 50.0 | mg |
| Sodium benzoate | q.s. | |
| Purified water | q.s. to 100 | ml |

The above ingredients were prepared and filled in the brown bottle of 100 ml to obtain solution by a conventional preparation method of solution.

### EXPERIMENTAL EXAMPLE 1

Whitening Effect of the extract of Pulsatilla Radix on Clinical Trial

Whitening effect was carried out with 20 volunteer women. Among them 10 of the women had freckles on their faces and remaining 10 women had liver spots on their faces.

10 women of freckles were divided into A, A′(A group), B B′(B group), C, C′(C group), D, D′(D group), E and E′ (E group). P100 was administered to A group; P400 to B group; P800 to C group; P1000 to D group, respectively and only the base was administered to E group. The methods of administrations were to rub on the places of freckles of the faces.

The same methods were applied to the women of liver spots. The groups were divided into G, G′(G group), H, H′(H group), I, I′(I group), J, J′(J group), K, L′(K group) and L, L′(L group).

The volumes of the composition and the base were each 500 mg per 5×5 cm². The numbers of administrations were 3 times a day. Before administrations, faces were washed. The administrations were continued for 3 weeks.

Three persons comparatively observed from the beginning of the clinical trials, discussed and judged effects on the base of criteria of cured, effective, slightly effective and non-effective of the whitening effects.

The results were shown on the Table 2.

TABLE 2
Whitening effects of the extract of Pulsatilla Radix on frecke and liver
spot of women:

| Freckes | | Liver spots | |
|---|---|---|---|
| A, A′(100 mg) | A: non effective | G, G′(100 mg) | G: non effective |
| | A′: non effective | | G′: non effective |
| B, B′(400 mg) | B: effective | H, H′(400 mg) | H: slightly effective |
| | B′: slightly | | H′: slightly effective |
| | effective | | |
| C, C′(600 mg) | C: cured | I, I′(600 mg) | I: cured |
| | C′: effective | | I′: cured |
| D, D′(80 mg) | D: cured | J, J′(800 mg) | J: cured |
| | D′: cured | | J′: cured |
| E, E′(1000 mg) | E: cured | K, K′(1000 mg) | K: cured |
| | E′: cured | | K′: cured |
| F, F′(base) | F: non effective | L, L′(base) | L: non effective |
| | F′ non effective | | L′: non effective |

Remarks:
Cured: Freckles or liver spots were disappeared.
Effective: Great parts of freckles or liver spots were disappeared.
Slightly effective: Some of freckles or liver spots were decreased.

As shown on the Table 2, P100 did not show effects on freckle group A,A′(100 mg) or liver spot group G,G′(100 mg); P400 showed slightly effective on freckle group B,B′(400 mg) or liver spot group H,H′(400 mg); More dosage group than 400 mg showed effective. More dosage group than 600 mg showed effective on all the groups in case of liver spots. More dosage group than 800 mg showed effective on all the groups in case of freckles.

### EXPERIMENTAL EXAMPLE 2

Whitening Effect of Injection on Clinical Trial

Five women having liver spots were selected and marked as A, B, C, D and E. Each 5.0 ml of the injection were injected to the women respectively for a day for 3 days successively and after that the same procedures carried out and ointment administered at the same time. Three women, A, B, C were cured 5 days after the administrations of injection and ointment and women D and E were effective.

A combined treatment of the administration of injection and ointment was more immediate effective than ointment treatment on skin without injection.

Clinical trials confirmed that the present composition has effectiveness. The effectiveness was carried out by the measuring method of inhibition of tyrosinase generally used in this field at the same time.

The PT and PA fractions inhibited 10% and 73% of activity of tyrosinaseat at 5 mg/ml respectively.

Pure ranunculin inhibited 51% of activity of tyrosinase

Deoxypodophyllotoxin inhibited 38% of activity of tyrosinase at 0.03 μg/ml. It was difficult that experiment at higher concentration could not be carried out because the material has very low solubility in water.

### EXPERIMENTAL EXAMPLE 3

Whitening Effect of Preparation Composed of Ranunculin, Deoxypodophylotoxin and SB365 on Clinical Trial

Pharmaceutical composition: 10 mg of ranunculin, 50 mg of deoxypodophyllotoxin, and 15 mg of SB365 were mixed well together with the base (19 g) to obtain pharmaceutical composition and the composition was rubbed on the area of liver spots three times a day till the time of curing.

Among five women (A,B,C,D and E), A and B were cured after 10 days; C was cured after 17 days; D was cured after 27 days and E was not cured, though the composition was slightly effective.

A composition from which SB365 which was not effective on tyrosinase was deleted was used. The composition were administered to five women (F,G,H,I,K) having liver spots. F and G were cured after 26 days; I was cured after 35 days; and J and K were the level of effectiveness.

From the result of the clinical trial, SB365 did not inhibit the activity of tyrosinase but have whitening effect. There is a possibility that SB 365 acts on melanocyte and interrupts the procedure of the migration of melanin to karatinocyte. An exact mechanism may be disclosed scientifically.

By adding the extract of the bark of the Ulmus macrocarpa, the extract of Ginseng Radix and/or the extract of Glycyrrhizae Radix as auxiliary ingredients to the preparation of Pulsatillae Radix having whitening effect, the combined composition can be anticipated to have merits of beauty arts, such as synergic effect of whitening action, skin-protection through bacteriocidal effect of saponin, skin-elutriation and skin-penetration effect, etc.

### EXPERIMENTAL EXAMPLE 4

Measurement of Inhibition Action Against Tyrosinase

Tyrosinase was purchased from Sigma Company. The enzyme was dissolved in 3,4-dihydrophenylalanine. Sodium phosphate buffer(0.1 M, pH 6.0), a substrate of enzyme and adjusted 1.6 mg/ml of concentration to obtain an enzyme solution. Sample was dissolved in sodium phosphate buffer and the undissolved residue was filtered out. 0.7 ml of the buffer, 0.2 ml of sample and 0.1 ml (15.7 unit/ml) of the enzyme solution were mixed and after 60 seconds, absorbance was measured at 475 nm by spectrophotometer.

By the following equation, inhibition ratio(%) of tyrosinase was calculated.
Inhibition ratio(%) of tyrosinase=[1-(S−B)/C]×100
wherein, S: Test tube having enzyme and sample,
B: Test tube having sample,
C: Test tube having enzyme.

### EXPERIMENTAL EXAMPLE 5

To each EAGLE MEM culture mediums containing 10% of foetus serum of cow were added each concentrations of the composition of Example 3 listed in Table 3 below and B-16 cells originated from mouse melanoma were inoculated to each culture mediums and were cultured under the condition of 37° C., 5% CO₂ for 5 days. The cells were dispersed with trypsin; centrifuged at 1,000 rpm for 5 minutes; collected and the melanoid degrees were measured macroscopically.

The criteria of judgement were as follows:
−: About same degree of not adding of the composition of Example 3,
+: Slightly whitening,
++: fairly whitening.

+++: mostly whitening.

TABLE 3

| | Amount of the | |
|---|---|---|
| | composition of Example 3 | |
| | (concentratiion, weight %) | Results |
| control | — | − |
| | 0.01 | + − |
| | 0.1 | ++ |
| | 0.3 | ++ |
| | 0.5 | ++ |
| | 0.7 | +++ |
| | 1.0 | +++ |

As ascertained from the above results, the composition of the present invention has excellent whitening effect on melanin originated from the mouse melanoma.

### EXPERIMENTAL EXAMPLE 6

On each inside parts of right brachiums of volunteers(heathy mem and women, each 15, total 30 persons), each 2×2 cm² parts were chosen ; the chosen parts were washed well with warm water; other parts than the chosen parts were masked with aluminum foil in order for ultraviolet ray to be irradiated only on the chosen parts; and with two FL20SBLB lamp(Toshiba Co., Ltd.) and FL20SE-30 lamp(Toshiba Co., Ltd.) at the same time, ultraviolet ray was irradiated the volume of 0.8×10 erg/cm³/time/day for successively 3 times. After irradiation, to the irradiated parts there applied the sample of below Table 4 three times a day(morning, noon and night). Evaluations were measured, after 3 weeks, the melanoid degrees macroscopically. The improvement degrees were evaluated as three degrees as excellent effective, effective and not effective.

TABLE 4

| Ingredients | concentration(weight part) |
|---|---|
| Composition of Example 3 | 0.5 |
| Polyoxyethylene(40)monostearate | 2.0 |
| Glycerolmonostearate | 5.0 |
| Stearic acid | 5.0 |
| Behenyl alcohol | 1.0 |
| Fluid paraffin | 1.0 |
| Glyceryltrioctanoate | 10.0 |
| 1,3-butylenglycol | 5.0 |
| Purified water | q.s. |

Other ingredients exempt 1,3-butylenglycol and purified water were dissolved by warming.(Oily state). Separately, 1,3-butyleneglycol and purified water were mixed and warmed to obtain a solution. The solution was added to the oil and the mixture was stirred; emulsified; cooled to obtain a vanishing cream.(Vanishing cream of the extract of Pulsatilla Radix).

Separately, A vanishing cream exempt the extract of Pulsatilla Radix was prepared and used as control.

The results are as follows:

TABLE 5

| ingredient | excellent effective | effective | not effective |
|---|---|---|---|
| ext. of P. Radix | 17(persons) | 10(persons) | 3(persons) |
| control | 0(persons) | 3(persons) | 27(persons) |

As ascertained from the above experimental results, the cosmetic composition comprising extract of Pulsatilla Radix of the present invention has excellent inhibiting effect against melanin formation.

### INDUSTRIAL APPLICABILITY

The present invention relates to a whitening cosmetic composition comprising the extract of Pulsatillae Radix and if necessary, one or more ingredient(s) selected from the group consisting ranunculin, deoxypodophyllotoxin and 3-O-α-L-ramnopyranosyl(1→2)-[β-D-glucopyranosyl(1→4)]-α-L-arabinopyranoside(SB365) extracted and isolated from the extract of Pulsatillae Radix as main ingredients and if necessary, more comprising one or more extracts selected from the group consisting the extract of the bark of the Ulmus macrocarpa, the extract of Ginseng Radix and the extract of Glycyrrhizae Radix as auxiliary ingredients. The present composition has an excellent whitening effect.

1. A whitening cosmetic composition comprising extract of Pulsatillae Radix as active ingredient.

2. A whitening cosmetic composition comprising extract of Pulsatillae Radix and one or more ingredient(s) selected from the group consisting ranunculin, deoxypodophyllotoxin and 3-O-α-L-ramnopyranosyl(1→2)-[β-D-glucopyranosyl(1→4)]-α-L-arabinopyranoside(SB365) extracted and isolated from the extract of Pulsatillae Radix as main ingredients.

3. A whitening cosmetic composition according to the claim 1 or the claim 2 comprising one or more extracts selected from the group consisting extract of bark of Ulmus macrocarpa, extract of Ginseng Radix and extract of Glycyrrhizae Radix as auxiliary ingredient(s).

4. A whitening cosmetic preparation comprising the composition of the claim 1 characterized in that said composition is mixed with conventional adjuvants and is prepared to a conventional preparation.
